(19) United States
(12) Patent Application Publication
(10) Pub. No.: US 2025/0185998 A1
(43) Pub. Date: Jun. 12, 2025

(54) MULTIMODAL PREDICTION OF PERIPHERAL ARTERIAL DISEASE RISK

(71) Applicants: Andrew R. Nimmich, Napa, CA (US); Ravi Dilip Patel, Pflugerville, TX (US)

(72) Inventors: Andrew R. Nimmich, Napa, CA (US); Ravi Dilip Patel, Pflugerville, TX (US)

(21) Appl. No.: 18/972,340

(22) Filed: Dec. 6, 2024

Related U.S. Application Data
(60) Provisional application No. 63/607,541, filed on Dec. 7, 2023.

(51) Int. Cl. A61B 5/00; A61B 5/02; A61B 5/022; A61B 5/0295; A61B 5/145; G16H 10/20; G16H 10/60; G16H 20/00; G16H 40/63; G16H 50/30

(52) U.S. Cl. CPC A61B 5/7275; A61B 5/02007; A61B 5/02225; A61B 5/0295; A61B 5/14542; A61B 5/6824; A61B 5/6829; A61B 5/7267; G16H 10/20; G16H 10/60; G16H 20/00; G16H 40/63; G16H 50/30

(57) ABSTRACT

A system leverages a multimodal model to predict peripheral arterial disease (PAD) risk. The system provides a dynamic questionnaire leveraging a language model to generate questions in response to user input. From the dynamic questionnaire, the system identifies user-specific risk factor(s) for PAD. The system also receives sensor data recorded by health sensor(s) including biometric signals of the user. The system applies a sensor classification model to the sensor data to output clinical data associated with a health state of the user. The system applies a multimodal PAD risk prediction model to the identified user-specific risk factors and the clinical data to output a PAD risk prediction indicating whether the user is at risk for PAD. Based on the outputs, the system generates and transmits one or more personalized recommendations for the user for treating or mitigating PAD risk and/or control instructions for controlling operation of the health sensor(s) and/or the medical device(s).

### CROSS-REFERENCE TO RELATED APPLICATIONS

The present application claims the benefit of and priority to U.S. Provisional Application No. 63/607,541 filed on Dec. 7, 2023, which is hereby incorporated by reference in its entirety.

### BACKGROUND

Medical practitioners presently diagnose peripheral arterial disease (PAD) based on patient chart information, patient examination, and test results performed by complex diagnostic equipment. Patients typically attend two to four visits to a medical facility for diagnosis that can be of significant cost and may take up to two months or more. For patients suffering from peripheral arterial disease, waiting through a traditional lengthy diagnosis process without receiving treatment can lead to loss of digits, limbs, and sometimes, in rare circumstances, the life of a patient. Moreover, the traditional screening paradigm applies generalized cutoffs and patient mobility restrictions for diagnosing PAD that may be ill-suited to individual patients. These challenges lead to severe under diagnosis and, consequently, missed treatment opportunities. Accordingly, there remains technical challenges to early prediction of PAD, such that treatment options can be implemented earlier, thereby saving life and limb.

### SUMMARY

An analytics system performs multimodal prediction of peripheral arterial disease (PAD) risk. The system pairs patient-specific risk factors with clinical data derived from biometric signals measured by wearable health sensors and/or imaging equipment to perform the multimodal prediction.

To identify the risk factors, the system provides a dynamic questionnaire to uncover information on the patient. The system leverages a language model to dynamically generate the questions, e.g., in response to user input. The language model may further refer to the patient's past medical history in generating relevant questions. In one or more embodiments, the language model may be a large language model trained on a large corpus of text. In one or more embodiments, the language model may be configured as a multimodal model trained on multimodal data (e.g., text, images, audio, etc.). In one or more embodiments, the language model may include an agentic model aimed at achieving one or more objectives, e.g., through a series of actions or questions.

To predict clinical data, the system receives sensor data recorded by health sensor(s) and/or imaging either being obtained in real time or obtained at an earlier date which may include biometric signals of the patient. The health sensor(s) may be wearable devices configured for at-home sensing of biometric signals. In one or more example implementations, the health sensor(s) may include a pair of pressure cuff devices coupled to different limbs of the patient to measure blood pressure at the different limbs. In other embodiments, the health sensor(s) may include an ultrasonic wearable patch or similar device, computed tomography angiography (CTA), magnetic resonance angiography (MRA), fluoroscopy, or any vascular imaging device capable of providing data on vascular health and perfusion. The system applies a sensor classification model to the sensor data to output clinical data associated with a health state of the patient. In the example above, the sensor classification model may be applied to the pressure readings recorded by the pair of pressure cuff devices, or data derived from the other imaging or sensing modalities, to output an ankle-brachial index (ABI) value or other metrics indicating a differential in vascular health or perfusion between a brachial artery and the lower peripheral artery, i.e., in the legs.

The system applies a multimodal PAD risk prediction model to the identified patient-specific risk factors and the clinical data to output a PAD risk prediction indicating whether the patient is at risk for PAD. Based on the outputs, the system generates and transmits one or more personalized recommendations for the patient for treating or mitigating PAD risk.

This multimodal PAD risk prediction methodology provides for a technical improvement in that a patient can perform a number of the screening procedures outside the clinic context. Such accessibility empowers more rapid PAD risk prediction than afforded by the traditional screening paradigms. Moreover, traditional screening may evaluate a patient's clinical data against global norms-which can lead to impersonalized predictions or diagnoses. The multimodal prediction combining the patient-specific risk factors and the clinical data empowers tailored predictions that more closely align with the patient's current health state. Furthermore, the personalized recommendations informed by the patient-specific risk factors and the clinical data can provide better tailored insights to treating or mitigating the PAD risk.

### BRIEF DESCRIPTION OF THE DRAWINGS

FIG. 1 illustrates a networked computing environment for multimodal prediction of peripheral arterial disease (PAD) risk, according to one or more embodiments.

FIG. 2 illustrates a block diagram representing an architecture of the analytics system of FIG. 1, according to one or more embodiments.

FIG. 3 is an illustrative flowchart of the multimodal prediction of PAD risk by the analytics system of FIG. 2, according to one or more embodiments.

FIG. 4 is a method flowchart of the multimodal prediction of PAD risk by an analytics system, according to one or more embodiments.

FIG. 5 is an example treatment recommendation workflow, according to one or more embodiments.

### DETAILED DESCRIPTION

The Figures (FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

### Networked Computing Environment

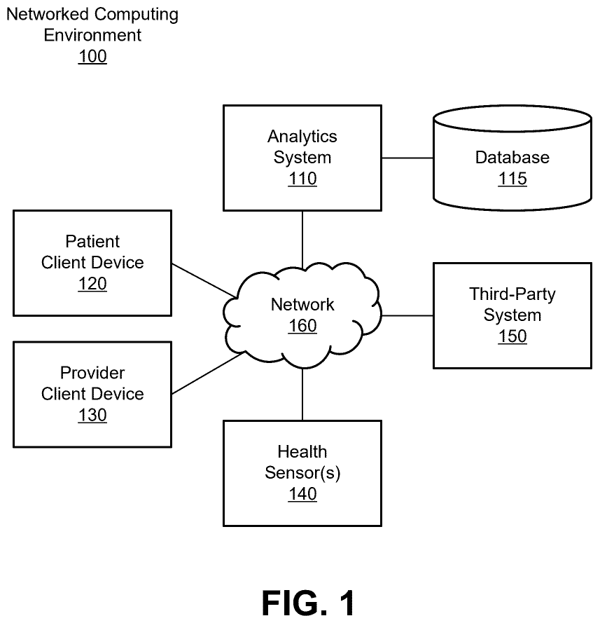

FIG. 1 illustrates a networked computing environment for multimodal prediction of peripheral arterial disease (PAD) risk, according to one or more embodiments. The computing environment 100 includes an analytics system 110, a database 115, a patient client device 120, a provider client device 130, one or more health sensor(s) 140, and a third-party system 150 connected via a network 160. Alternative embodiments may include additional, fewer, or different entities than those listed in FIG. 1. In some embodiments, any one component may be combined with other components, or any component may be distributed across a set of computing devices. In other embodiments, the networked computing environment 100 may be expanded to multimodal prediction of other diseases, by leveraging the dynamic questionnaire and the prediction of clinical data from biometric signals captured as sensor data.

The analytics system 110 performs analyses, e.g., including multimodal prediction of PAD risk. The analytics system 110 includes one or more modules and one or more models for analyzing the data received from the other entities in the networked computing environment 100. For example, the analytics system 110 receives user input from the patient client device 120 and/or sensor data from the health sensor(s) 140. The analytics system 110 performs analyses on the user input and the sensor data to determine the PAD risk. The analytics system 110 may report the PAD risk to the patient client device 120 and/or the provider client device 130. Based on the determined PAD risk, the analytics system 110 may also generate recommendations for further steps in a diagnostic work-up, for a treatment plan, or for further sensing via the health sensor(s) 140.

In some embodiments, to perform multimodal prediction of PAD risk and/or to generate follow-on recommendations based on the predicted PAD risk, the analytics system 110 leverages one or more machine-learning models. In some embodiments, the analytics system 110 leverages a set of machine-learning models. The analytics system 110 may leverage a language model to generate a dynamic questionnaire to identify patient-specific risk factors. The analytics system 110 may also leverage a sensor classification model configured to input sensor data, e.g., from the health sensor(s) 140, and/or health data and to output clinical data representing the patient's medical state. The analytics system 110 may further leverage a fusion model configured to input the patient-specific risk factors, i.e., obtained from the dynamic questionnaire, and the clinical data, i.e., output by the sensor classification model, and to output a PAD risk prediction. The PAD risk prediction may be a multiclass label, e.g., five categories of PAD risk. The PAD risk prediction may, alternatively or additionally, be a numerical value indicating the level of risk.

The analytics system 110 may generate recommendations specific to the patient based on the PAD risk prediction, the patient-specific risk factors, the clinical data, or some combination thereof. The analytics system 110 may leverage a list of available treatment actions. The analytics system 110 may maintain heuristics to determine when each treatment action may be recommended to a user. The analytics system 110 may identify one or more treatment actions to recommend to the patient based on the PAD risk prediction, the patient-specific risk factors, the clinical data, or some combination thereof. In some embodiments, the analytics system 110 may provide recommendations to the provider client device 120, e.g., for further investigation by the healthcare provider. In one or more embodiments, the analytics system 110 may further leverage the language model to identify recommendations to provide to the patient and/or the healthcare provider.

The analytics system 110 may be implemented using cloud processing and storage technologies, on-site processing and storage systems, virtual machines, other technologies, or a combination thereof. For example, in a cloud-based implementation, the analytics system 110 may include multiple distributed computing and storage devices managed by a cloud service provider. The various functions attributed to the analytics system 110 are not necessarily unitarily operated and managed, and may comprise an aggregation of multiple servers responsible for different functions of the analytics system 110 described herein. In this case, the multiple servers may be managed and/or operated by different entities. In various implementations, the analytics system 110 may comprise one or more processors and one or more non-transitory computer-readable storage mediums that store instructions executable by the one or more processors for carrying out the functions attributed to the analytics system 110.

The database 115 stores data used by the analytics system 110, e.g., for the multimodal prediction of PAD risk and/or the generation of follow-on recommendations. In one or more embodiments, the database 115 may store data on the patient, subject to the patient opting in to the use thereof. The patient data may include medical history of a patient, result of prior analyses by the analytics system, treatment recommendations, lifestyle of the patient, previously obtained vascular imaging data, other data related to multimodal prediction of PAD risk, or some combination thereof. In some embodiments, the database 115 stores data associated with historical vascular or other relevant procedures performed on a patient.

In some embodiments, the database 115 may include one or more publicly available datasets such as the Society for Vascular Surgery (SVS) Vascular Quality Initiative (VQI) database. The database 115 may furthermore include various public or non-public data from electronic health records (EHR) systems, clinical trials, or other sources to guide PAD risk prediction.

In some embodiments, the database 115 may include one or more cloud-based data sources and/or one or more locally accessible data sources. In some embodiments, the database 115 may comprise a centralized repository that may aggregate data from multiple different sources. In other embodiments, the database 115 may refer to two or more disparate data sources that may be managed by different entities and may be independently accessed by the analytics system 110. The database 115 may be accessible via an application programming interface (API) or may enable data to be downloaded via a web browser or other application.

The database 115 may incorporate various structured or unstructured information. For example, structured information may include data organized into predefined fields while unstructured data sources may include articles or other media. Examples of structured data fields in the database 115 may include, for example, demographic characteristics of patients (e.g., race, age, location, height, weight, body mass index (BMI), educational attainment, etc.), health histories of patients (e.g., patient symptoms, diagnosed conditions, prescribed medications, dietary habits, cigarette use, alcohol use, drug use, etc.), laboratory and/or imaging results from other facilities, information about specific performed procedures (e.g., type of procedure, facilities where procedures are performed, physician performing the procedure, equipment used, etc.), outcomes of procedures (e.g., patient recovery information, recommended follow up treatments, etc.) or other data that may be relevant to analyzing patterns in peripheral arterial disease, surgical risk, and/or related areas of vascular health. The database 115 may furthermore store various contextual information relating to the quality of data, sources of the data, and computation methods.

The database 115 may be organized into records that each correspond to specific performed procedures relating to peripheral arterial disease and may include values associated with different fields representing different features. Each data record is not necessarily complete. Some records may include values for every field, while other records may be missing information for one or more fields. Values associated with different fields may be represented in different ways for different fields. For example, data may be presented as numeric values, text values, dates, or coded values.

The patient client device 120 is a computing device in use by the patient. The patient client device 120 can execute a user interface enabling access to various functionality provided by the analytics system 110. In one or more embodiments, the user interface may be embodied in an application installed on the patient client device 110 or may comprise a web-based application accessible via web browser. The user interface of the patient client device 120 may include input elements for various entry of data. The user interface may also present information provided by the analytics system 110. In various embodiments, the patient client device 120 may be embodied, for example, as a mobile phone, a tablet, a laptop computer, a desktop computer, a gaming console, a head-mounted display device, or other computing device. The patient client device 120 may be used by a patient to provide user input for generating a PAD risk prediction or another analysis associated with PAD. The patient client device 120 could furthermore include a computing device linked to an EHR system and/or hospital outreach platform that may provide a communication pathway to the analytics system 110, e.g., via an API. The device may also have an interface within a third-party charting system that allows for automated PAD risk prediction based solely on chart data that can include real time biometric data when available.

The provider client device 130 is a computing device used by a healthcare provider to a patient. The healthcare provider may be a physician or other licensed professional providing care to the patient. The healthcare provider uses the provider client device 130 to view results of analyses by the analytics system. The healthcare provider may also use the provider client device 130 to communicate with the patient. For example, the interfaces of the patient client device 120 and the provider client device 130 may include a communication platform for back and forth communication between the patient and their healthcare provider. Via the communication platform, for example, the provider client device 130 may provide results of the analyses performed by the analytics system 110. The provider client device 130 may also transmit treatment recommendations from the provider, or follow-on steps for completing a diagnostic work-up. The provider client device 130 may furthermore include a computing device linked to an EHR system that may enable various configuration settings and other administrative functions associated with its integration with the analytics system 110. The interface of the provider client device 130 may be embodied as an application installed on the provider client device 130 or may comprise a web-based application accessible via web browser. In various embodiments, the provider client device 110 may be embodied, for example, as a mobile phone, a tablet, a laptop computer, a desktop computer, a gaming console, a head-mounted display device, or other computing device.

The health sensors 140 are devices configured to sense biometric signals of a patient. The health sensors 140 may be wearable devices configured for continuous or periodic sensing of biometric signals. In one or more embodiments, the health sensors 140 may wirelessly connect to the patient client device 120, e.g., via Bluetooth, WiFi, or any other wireless or wired communication protocol. In some embodiments, the health sensors 140 may be controlled in part by instructions by the patient client device 120 and/or the analytics system 110. In such embodiments, the analytics system 110 and/or the patient client device 120 may provide a control signal that causes the one or more health sensors 140 to initiate a session for sensing the biometric signals and/or provide real-time imaging of the blood vessel.

In one or more embodiments, the health sensors 140 may include a pair of devices that collectively measure ankle brachial index (ABI) values or other relevant perfusion data to aid in diagnosis. For example, a first ring device is coupled to a finger and a second device is coupled to a toe. The devices may include volume plethysmography sensors to sense changes in blood volume. The devices may additionally include various supporting circuitry such as a controller, battery, and transmitter. The respective devices each obtain sensor waveforms over a sensing period. The devices may measure and record the biometric signals. The devices may transmit the biometric signals to the analytics system 110 and/or the patient client device 120. The analytics system 110 and/or the patient client device 120 may compute ABI values based on the ratios between the measurements from the respective devices. The ABI values may be used for various diagnostic tests as described below.

In one or more embodiments, other health sensors 140 may include a pulse oximeter for measuring blood oxygen saturation level and/or a pulse rate. The pulse oximeter includes a light emitter and a photodetector. The light emitter and the photodetector are configured to contact the patient's body (e.g., the skin). The light emitter emits light in a specific wavelength, and the photodetector detects reflected light. The pulse oximeter may compute the blood oxygen saturation level based on a difference between the emitted light and the reflected light. In one or more embodiments, other health sensors 140 may include a blood pressure monitor for measuring blood pressure and/or a pulse rate. The blood pressure monitor may include a pressure cuff that fits around the patient's limb. The cuff is inflated to provide uniform pressure to the limb. The blood pressure monitor measures the blood pressure that restores the blood flow through the limb, i.e., counteracting the pressure in the cuff. In one or more embodiments, other health sensors 140 may include a glucose monitor for measuring blood glucose levels. The glucose monitor can sense an amount of glucose in a blood sample. In other embodiments, the other health sensors 140 may include an electrocardiogram (EKG) device for measuring electrical activity of the heart. The EKG device leverages patches to measure electric differentials across different vectors of the heart. In other embodiments, the other health sensors 140 may include an ultrasonic imaging device for measuring volumetric blood flow through one or more blood vessels in the body. The ultrasonic device leverages ultrasound waves to estimate directional flow of fluids, i.e., leveraging the Doppler effect. In the ultrasonic device, ultrasonic sound waves are transmitted into the patient's body. An acoustic sensor measures any reflected sound waves. The difference between the frequencies of the emitted sound waves and the frequencies of the captured sound waves can be used to compute the volumetric blood flow. The ultrasonic sound waves may be emitted and received via a traditional ultrasound probe, an ultrasound patch, or other embodiments that may be utilized to effectively produce ultrasonic data.

The third-party system 150 may provide additional data, e.g., for use by the analytics system 110. The third-party system 150 may be an online platform of a medical journal, providing updated insights and research relating to various health issues. For example, as novel research for peripheral arterial disease (PAD) is published in the medical journal, the analytic system 110 may obtain the publication to inform the multimodal prediction of PAD risk and/or the generation of follow-on recommendations. In other embodiments, the third-party system 130 may be associated with patient records stored by a healthcare system, e.g., a hospital. Contingent upon approval by the patient to disclose data, the third-party system 130 may provide data relating to a patient's medical history to the analytics system 110, e.g., for use in the multimodal prediction of PAD risk and/or the generation of follow-on recommendations. The third-party system 130 may be embodied as a computing device or server, capable of storing data maintained by the third-party system. In various embodiments, the provider client device 110 may be embodied, for example, as a mobile phone, a tablet, a laptop computer, a desktop computer, a gaming console, a head-mounted display device, or other computing device.

The one or more networks 108 provides communication pathways between the database 115, the analytics system 110, the provider client device 130, and/or the user clients 110. The network(s) 108 may include one or more local area networks (LANs) and/or one or more wide area networks (WANs) including the Internet. Connections via the one or more networks 108 may involve one or more wireless communication technologies such as satellite, WiFi, Bluetooth, or cellular connections, and/or one or more wired communication technologies such as Ethernet, universal serial bus (USB), etc. The one or more networks 108 may furthermore be implemented using various network devices that facilitate such connections such as routers, switches, modems, firewalls, or other network architecture.

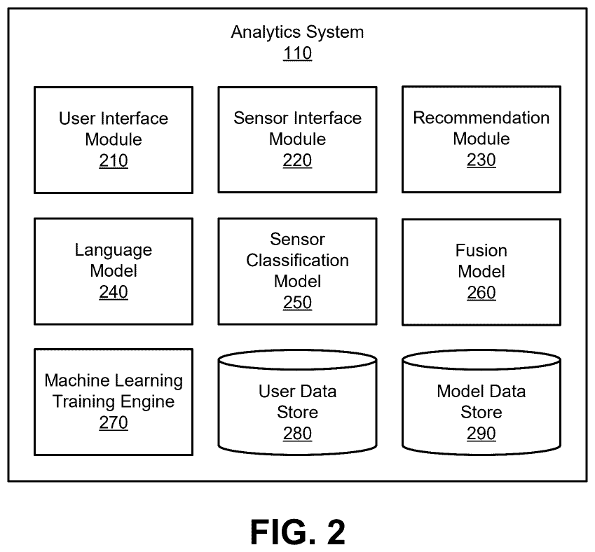

FIG. 2 illustrates a block diagram representing an architecture of the analytics system 110 of FIG. 1, according to one or more embodiments. The analytics system 110 may include one or more computer processors and one or more storage mediums with encoded instructions executable by the computer processors. The analytics system 110 includes a user interface module 210, a sensor interface module 220, a recommendation module 230, a language model 240, a sensor classification model 250, a fusion model 260, a machine-learning training engine 270, a user data store 280, and a model data store 290. In other embodiments, the analytics system 110 may include additional, fewer, or different components than those listed in the block diagram. In other embodiments, functionality of the various components may be disparately distributed than described herein.

The user interface module 210 generates one or more user interfaces for use on the client devices. The user interface module 210 may generate the user interfaces to include components for presenting content to the users. For example, one component presents a dynamic questionnaire that asks one or more questions with an input element for providing a response to the questions. In another example, one component presents a messaging platform for empowering the patient to communicate with their healthcare provider. In another example, one component can present results of analyses performed by the analytics system 110. In yet another example, one component can present treatment recommendations by the analytics system 110 and/or the provider client device 130. In an example operation, the user may input various information via the user interface on the patient client device 120 that is in communication with the user interface module 210, such as inputs describing a patient's symptoms, demographic information, clinical history, or other information.

In some embodiments, the user interface module 210 generates a dynamic questionnaire including a series of questions with an input option to provide structured input of data. Questions may be presented for various input forms such as multiple choice, true/false, or text-based inputs. The user interface may utilize various input elements such as radio buttons, drop-down lists, multi-select checkboxes, or freeform text boxes. The user interface module 210 may leverage a language model 240 trained to generate questions for the dynamic questionnaire. The language model 240 may be informed by the patient's medical history and responses to earlier questions in the dynamic questionnaire. Based on these inputs, the language model 240 may identify follow-on questions to query the patient, e.g., via the user interface. The follow-on questions are presented by the user interface module 210 via the user interface presented on the patient client device 120.

In some embodiments, the user interface module 210 may include data prompts to request information relating to symptoms and medical history such as the following:

### Symptoms:

Does the patient have leg pain?
If so, is it worse with exertion?
If it is worse with exertion, how far can the patient walk before experiencing the pain? (The patient may furthermore be promoted to complete a walking impairment questionnaire such as that recommended by the SVS).
Does the patient's pain prevent them from doing things to live their life as they would like? (lifestyle limiting claudication)
Does the patient have it at rest?
Is the pain worse with a certain position?
Is the pain worse with standing upright, or lying flat?
Does the patient have a wound?
If so, does the wound have drainage?
Approximately, how large is the wound and where is it located. Instruct the patient to take a photo of the wound and upload it to the application.
What are the patient's associated symptoms? (numbness, tingling)
What color are the patient's feet?
Does the patient have hair growth on their feet?

### Medical History:

### Current PAD History:

Wounds that do not heal?
Leg pain that gets worse when the patient walks yet is relieved with rest?
Leg pain/cramps/restless legs at rest?
Tried to exercise to help with the leg pain? How long?
Did it help relieve the pain?
What part of the leg hurts?
Does leg pain interfere with work or daily activities?
How far can the patient ambulate before the pain starts?
What pain medications have you taken for the leg symptoms?

### Past PAD History:

Has the patient ever had lower extremity bypass surgery?
Has the patient had prior lower extremity angioplasty or stenting?
Has the patient had prior amputation(s) of the legs, feet or toes?
Has the patient ever had a Heart Attack or Stroke?
Does the patient have High Blood Pressure?
Does the patient have Diabetes?
Does the patient have High Cholesterol?
Is the patient pregnant (If female of less than 55 years of age)?

### Past Surgical History:

What past surgeries and/or procedures has the patient had?

### Lifestyle History:

Smoker? Pack Per Day? Years?
Alcohol Use?
Drug Use?
Diet?
Exercise?

### Allergies

List allergies

In one or more embodiments, the user interface module 210 may present the dynamic questionnaire as a conversation with an artificial intelligence (AI) agent. For example, the AI agent may be trained to simulate an interaction with a healthcare provider, that is starting with an introductory greeting, before asking about current symptoms. As the user provides responses or engages in the conversation, the AI agent may leverage the language model 240 to identify follow-on questions to ask the patient. For example, if the patient explains that they're having some worsening leg pain, the AI agent, leveraging the language model 240, may inquire further into the leg pain. The AI agent may be tasked with uncovering certain information that can inform a patient's PAD risk factors.

In one or more example implementations, upon completing the dynamic questionnaire, the user interface module 210 patient may be prompted to perform one or more diagnostic evaluations such as an Ankle-Brachial-Indices (ABI) evaluation. The ABI evaluation may be performed using traditional equipment in a medical facility or may be performed using the wearable devices 130 as described above.

The user interface module 210 may furthermore facilitate presentation of various outputs from the analytics system 110. For example, the user interface module 210 may output a predicted PAD risk score, a summary of automated diagnosis, recommendations, or some combination thereof. The outputs may be in the form of a percentage risk, a numerical score (e.g., on a scale of 1 to 10), a risk category (e.g., high, medium, low), or a combination thereof. In an embodiment, the user interface of the patient client device 120 may furthermore present various treatment recommendations or contextual information associated with the predicted diagnosis.

In some embodiments, the PAD risk may be classified into one of the following categories:
0=No intervention, no follow-up necessary
1=Surgical intervention (findings confirmed with surgeon prior to starting procedure)
2=Conservative management with repeat assessment in 6 months (definitions below)

Although the user interface module 210 is illustrated as component of the analytics system 110 in FIG. 2, all or a subset of the functions of the user interface module 210 may instead be executed on the patient client device 120. For example, the patient client device 120 may download an application from the analytics system 110 that includes all or some of the functions of the user interface module 210. The patient client device 120 may locally execute instructions associated with these functions.

The sensor interface module 220 receives biometric signals sensed by the one or more health sensors 140. The signals may be received from the patient client device 120 in communication with the health sensors. The signals may be in raw form as measured by the health sensors 140. The sensor interface module 220 preprocesses the signals for downstream analysis. In one or more embodiments, the sensor interface module 220 determines features from the biometric signals for input into the sensor classification model 250 to output clinical data on the patient. The biometric signals may be stored in the user data store 280.

In one or more embodiments, the sensor interface module 220 may generate control signals for controlling operation of the health sensors 140. For example, the sensor interface module 220 may, based on a predicted PAD risk or diagnosis by a healthcare provider, generate a control signal to perform follow-on sensing by the health sensors 140. The sensor interface module 220 may transmit the control signals to the health sensors 140 to cause the health sensors 140 to perform the sensing, i.e., in an automated manner. In other embodiments, the sensor interface module 220 may generate control signals for controlling operation of any other medical devices in communication with the analytics system 110. For example, a medical device aiding in providing therapy to the patient may be controlled, at least in part, via the control signals generated by the sensor interface module 220.

The recommendation module 230 determines one or more recommendations based on results of analyses by other components of the analytics system 110. For example, the recommendation module 230 may input a predicted PAD risk, a diagnosis of PAD, patient-specific risk factors, clinical data, past medical history, past treatment, or some combination thereof, to identify one or more recommendations. As noted elsewhere, the recommendations may include one or more treatment recommendations, follow-on steps for diagnostic work-up, or recommendations to perform additional biometric sensing. The recommendation module 230 may also leverage the language model 240 to identify the recommendations. As such, the language model 240 may identify recommendations from a plurality of possible recommendations based on the input data. For example, if the patient's past treatment history indicates that one treatment option is ineffective or minimally effective for the particular patient, the recommendation module 230, e.g., leveraging the language model 240, may exclude such treatment option in favor of other treatment options that have been effective or have not been yet tried. In one or more embodiments, the recommendation module 230 may provide a report of the analysis results with one or more recommendations to the healthcare provider, such that the healthcare provider can provide a formal diagnosis and/or treatment recommendation.

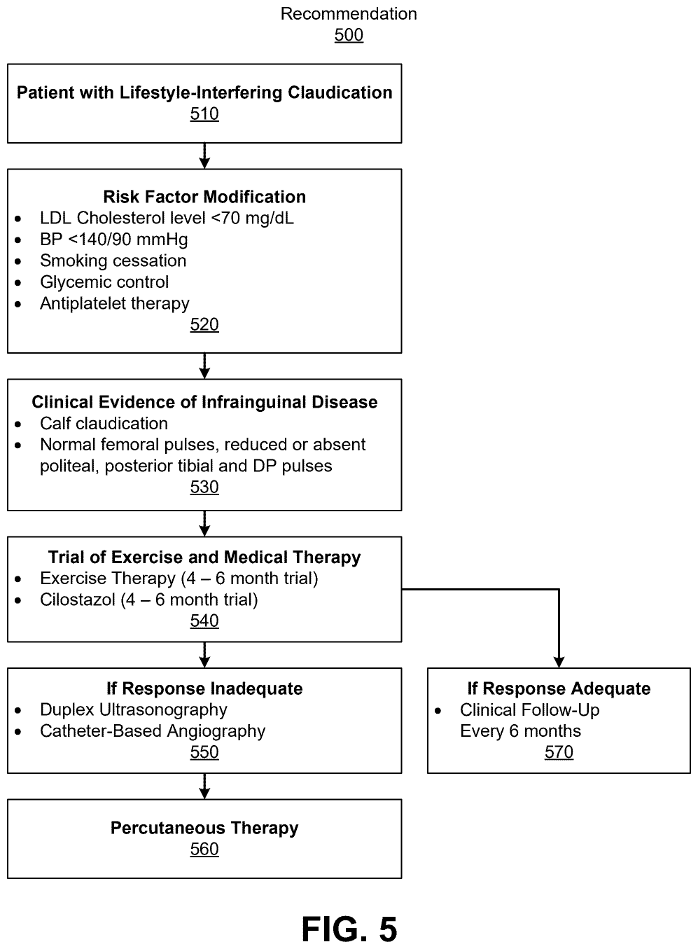

The recommendation module 230 may output a recommended treatment based on a PAD risk predicted by the analytics system 110. For example, for a patient with a risk score of 1 (surgical intervention recommended), treatment may be based on the patient's ABI, patient-specific risk factors and medical history, and any symptoms identified via the dynamic questionnaire. For example, a patient with an ABI<0.9, one or more risk factors (e.g., history of smoking, diabetes, hyperlipidemia/high cholesterol, high blood pressure, or over age 65), and one or more of the symptoms (wound and/or pain at rest of the lower extremities), then the recommendation module 230 may recommend a referral for surgical evaluation and diagnostic arteriogram. For a patient with a risk score of 2 (conservative management), an example treatment plan is further described in FIG. 5.

Alternatively, recommendations may be generated based on one or more machine-learning models trained to generate treatment recommendations. Here, the recommendation module 230 may assess various candidate treatment options to predict likelihoods success and recommend treatment with the highest likelihood.

The language model 240 is a machine-learning model configured to perform natural language processing (NLP) tasks. In one or more embodiments, the language model 240 is configured to generate a dynamic questionnaire for identifying patient-specific risk factors, e.g., in predicting PAD risk or diagnosing PAD in a patient. The language model 240 may be guided with objectives to be achieved in the dynamic questionnaire. For example, the objectives might specify specific information to uncover from the patient. The objectives may be structured in a decision tree, for example, a first branch may dive into physical symptoms, then a second branch may dive into lifestyle questions, before a third branch dives into any other miscellaneous health issues. The decision tree may be dynamic as well, shifting between patients and/or sessions with a patient. The dynamic nature of the decision tree may aid in the dynamic organization of questions provided by the dynamic questionnaire.

The language model 240 is configured to input a prompt and to output a response based on the prompt. The prompt generally includes instructions for performing a NLP task. The prompt may further include data or other context that constrains the NLP task. The prompt and/or the response may also be multimodal. In one example, the prompt at the start of the dynamic questionnaire may be:
“Generate a greeting and an open-ended question that asks the patient of any health updates.”
   The prompt is provided to the language model 240 for execution, which may yield the response:
“Hi, good morning! How are you feeling in today?”
   As the patient provides a response, the user interface module 210 may include the patient's input into follow-on prompts to the language model 240.

The language model 240 may further refer to data stored in the user data store 280, e.g., the patient's medical history. One example follow-on prompt may be:
“Based on the patient's response, ‘I'm having leg pain.” please generate a follow-on question that further inquiries into the details of the symptom.”
   Correspondingly, upon execution, the language model 240 may output the following response:
“I understand that you're feeling some leg pain. Could you explain further where the pain is felt and what level the pain is at, on a scale of 1 to 10?”
   The user interface module 210 and the language model 240 could proceed accordingly to uncover information for as many objectives as feasible. If certain information is uncertain or unknown, the user interface module 210 may identify the missing objectives, e.g., which may be provided to the healthcare provider for further investigation.

In one embodiment, the language models are large language models (LLMs) that are trained on a large corpus of training data to generate outputs for the NLP tasks. An LLM may be trained on massive amounts of text data, often involving billions of words or text units. The large amount of training data from various data sources allows the LLM to generate outputs for many tasks. An LLM may have a significant number of parameters in a deep neural network (e.g., transformer architecture), for example, at least 1 billion, at least 15 billion, at least 135 billion, at least 175 billion, at least 500 billion, at least 1 trillion, at least 1.5 trillion parameters.

Since an LLM has significant parameter size and the amount of computational power for inference or training the LLM is high, the LLM may be deployed on an infrastructure configured with, for example, supercomputers that provide enhanced computing capability (e.g., graphic processor units) for training or deploying deep neural network models. In one instance, the LLM may be trained and deployed or hosted on a cloud infrastructure service. The LLM may be pre-trained by the analytics system. An LLM may be trained on a large amount of data from various data sources. For example, the data sources include websites, articles, posts on the web, and the like. From this massive amount of data coupled with the computing power of LLM's, the LLM is able to perform various tasks and synthesize and formulate output responses based on information extracted from the training data.

In one embodiment, when the machine-learned model including the LLM is a transformer-based architecture, the transformer has a generative pre-training (GPT) architecture including a set of decoders that each perform one or more operations to input data to the respective decoder. A decoder may include an attention operation that generates keys, queries, and values from the input data to the decoder to generate an attention output. In another embodiment, the transformer architecture may have an encoder-decoder architecture and includes a set of encoders coupled to a set of decoders. An encoder or decoder may include one or more attention operations. While a LLM with a transformer-based architecture is described as a primary embodiment, it is appreciated that in other embodiments, the language model can be configured as any other appropriate architecture including, but not limited to, long short-term memory (LSTM) networks, Markov networks, BART, generative-adversarial networks (GAN), diffusion models (e.g., Diffusion-LM), and the like.

The sensor classification model 250 is configured to input features from sensor data recorded by the health sensors 140 to output predictions on clinical data representing a health state of the patient. The sensor classification model 250 may be trained as a machine-learning classification model. The sensor classification model 250 may be a neural network, a transformer-based model, a decision tree model, a support vector machine, or some combination thereof. In one or more embodiments, the sensor classification model 250 may be configured with a plurality of submodels, each submodel being trained and configured to output one type of clinical data based on the input features from the sensor data. In some embodiments, the submodels may be configured to input a particular type of sensor data features, e.g., features from the pressure cuff differential readings—the ABI values. Each submodel may be trained independently from the other submodels. In other embodiments, the submodels may be trained together, with information being shared across the submodels. In such embodiments, intermediary predictions are input or shared across submodels.

The fusion model 260 is configured to input the clinical data output by the sensor classification model 250 and the patient-specific risk factors identified from the dynamic questionnaire to output a prediction on PAD risk. The prediction on PAD risk may comprise a diagnosis for PAD. In one or more embodiments, the PAD risk prediction may be binary, i.e., whether the patient is at risk or not. In other embodiments, the PAD risk prediction may be a multiclass label, e.g., multiple categories of varying PAD risk. In yet other embodiments, the PAD risk prediction may be a scalar value representing the degree of PAD risk, e.g., a higher scalar value may reflect higher risk, as in a more aggressive form of PAD, whereas a lower scalar value may reflect lower risk, as in a less aggressive form of PAD. The fusion model may be trained as a machine-learning model. In one or more embodiments, the fusion model may also be architected with two sequential submodels. A first submodel is a binary classifier to predict whether the patient, based on the clinical data and the patient-specific risk factors, is at risk of PAD or not. Pending a positive indication of being at risk of PAD, a second submodel configured as a multiclass classifier or regression model is configured to predict a degree of PAD risk. The fusion model 260 may provide both the binary prediction and the multiclass or scalar value prediction.

The machine-learning training engine 270 trains one or more of machine-learning models used by the analytics system 110. The machine-learning training engine 270 trains each of the one or more machine-learning models with one or more training datasets. For example, the machine-learning training engine 270 may apply a supervised machine learning algorithm to the training dataset to learn a set of model parameters (e.g., weights) for classifying a set of input features based on respective statistical similarities to the training datasets. The classification result may be expressed in terms of a likelihood value (e.g., a value between [0, 1]) or a score on a predefined risk score scale. Here, a higher likelihood value represents a higher likelihood of peripheral arterial disease while a lower likelihood value represents a lower likelihood of peripheral arterial disease. In example implementation, the training module 220 may employ machine learning techniques such as logistic regression, random forest, gradient boosting, or neural networks (such as recurrent neural networks (RNNs), convolutional neural networks (CNNs), etc.). In some embodiments, the machine-learning training engine 270 may retrain the one more machine-learning models as corrections to the predictions by the machine-learning models are provided, e.g., by a healthcare provider. In an unsupervised manner, the machine-learning training engine 270 may leverage a training dataset without ground truth labels to identify emergent or latent patterns in the features of the training dataset. For example, a machine-learning clustering algorithm can cluster training samples with similar features together, agnostic of any ground truth labels for the training samples.

In one or more embodiments, the machine-learning training engine 270 trains the language model 240 as a large language model with a large corpus of text. The machine-learning training engine 270 trains the language model 240 to understand language by reviewing the syntax, relations between words, grammar, etc. within the large corpus of text. To tailor the language model 240 to perform certain natural language processing (NLP) tasks, the machine-learning training engine 270 may tune the language model 240 with a focused training dataset relevant to the certain NLP tasks being trained to perform. In one or more embodiments, the machine-learning training engine 270 may train the language model 240 as a multimodal model with different modalities of data. The machine-learning training engine 270 obtains datasets for each modality, and leverages such datasets to generate training examples for training of the language model 240. In such embodiments, the language model 240 may include sub-models configured specifically for one or more of the modalities. For example, the language model 240 may include a convolutional neural network for image processing, to extract features from input images. In another example, the language model 240 may include a diffusion model for generating images, etc. In one or more embodiments, the machine-learning training engine 270 may configure the language model 240 to include an agentic model (also referred to as an agent-based model). The agentic model may be optimized to achieve a set of objectives by identifying steps towards achieving the objectives. To train the agentic model, the machine-learning training engine 270 may score steps taken by a hypothetical agent based on an objective function. Strategies that maximize the objective function's score guide the training process.

According to one or more embodiments, to train the language model 240 to generate the dynamic questionnaire, the machine-learning training engine 270 may obtain focused training data comprising questionnaires or conversations between healthcare providers and their patients in diagnosing or treating PAD. The text transcripts of such conversations may be fed into the language model 240 to attune the model to generating a dynamic questionnaire when interfacing with a patient. In one or more embodiments, the machine-learning training engine 270 may also train the language model 240 (or another language model) to generate recommendations for treating PAD based on a patient's PAD risk prediction or PAD diagnosis. In such embodiments, the machine-learning training engine 270 may obtain past treatment recommendations from healthcare providers for patient's diagnosed with PAD, which may further indicate the severity or risk level of their PAD.

In one or more embodiments, the machine-learning training engine 270 may obtain feedback to the responses generated by the language model 240. For example, a healthcare provider may provide feedback that the questions were misworded or there were gaps of information that were not queried. Such feedback can be leveraged by the machine-learning training engine 270 to fine tune the language model. As another example, a patient may provide feedback on recommendations by the language model 240. For example, the patient may reject certain types of treatment recommendations (e.g., exercise or medication). In response, the machine-learning training engine 270 may generate additional training samples based on this feedback to tailor recommendations output by the language model 240.

In one or more embodiments, the machine-learning training engine 270 trains the sensor classification model 250 with labeled clinical data. In one or more embodiments, the machine-learning training engine 270 may train the sensor classification model 250 in a supervised manner. Accordingly, the machine-learning training engine 270 obtains sensor data, e.g., from clinical environment settings, with clinical annotations as ground truth labels of clinical data. For example, a healthcare provider may review a patient's ABI values to classify whether the patient has PAD. The machine-learning training engine 270 may obtain numerous such training samples where a healthcare provider has made a formal diagnosis based on the ABI values as measured by the cuff devices. The healthcare providers diagnosis is a ground truth label, to supervise training of the sensor classification model 250. To train the model, the machine-learning training engine 270 feeds the features of the sensor data (e.g., the ABI values) into the model to predict the clinical label (i.e., the clinical data). Based on an error between the prediction and the ground truth labeling, the machine-learning training engine 270 may adjust model parameters to minimize the error. In embodiments with submodels, the machine-learning training engine 270 may curate training datasets to independently and specifically train each submodel.

In one or more embodiments, the machine-learning training engine 270 trains the fusion model 260 in a supervised manner with diagnoses of PAD by healthcare providers. The PAD diagnoses may serve as training samples. Each PAD diagnosis may be paired with the patient's health status leading up to that PAD diagnosis. For example, each training sample further specifies the patient-specific risk factors identified by a healthcare provider during an in-person screening, and clinical data as measured by one or more health sensors 140. To train the model, the machine-learning training engine 270 inputs features representing the risk factors and the clinical data to output a prediction of PAD risk or PAD diagnosis. The machine-learning training engine 270 may compute an error between the prediction and the ground truth diagnosis. The machine-learning training engine 270 adjusts parameters of the model to minimize the error, thereby yielding a trained machine-learning model. In embodiments with submodels, the machine-learning training engine 270 may curate training datasets to independently and specifically train each submodel.

The user data store 280 stores data relating to users of the analytics system 110. For example, the user data store 280 may store patient data for each patient. The patient data may include past medical history, past treatment plans and/or their determined efficacy, past symptoms, past diagnoses, past analysis results by the analytics system 110, sensor data recorded by the health sensors 140 and received by the analytics system 110, questionnaire response, or some combination thereof. In one or more embodiments, the user data store 280 may store healthcare provider data for each healthcare provider. For example, the user data store 280 may store preferences by the healthcare provider, past diagnoses made by the healthcare provider, past treatment recommendations made by the healthcare provider, or some combination thereof. For example, if one healthcare provider typically recommends a more conservative treatment plan, the analytics system 110 may leverage the historical recommendations to tailor treatment recommendations, e.g., as determined by the recommendation module 230 optionally leveraging the language model 240, to bias towards the historical recommendations of the healthcare provider. In one or more embodiments, the user data store 280 is a component of the database 115.

The model data store 290 stores the one or more machine-learning models generated by the training module 220. The model data store 290 may store any statistical distributions or metrics thereof for use by the models. The model data store 290 may also the architecture of the one or more models, the learned parameters (i.e., weights) of the one or more models, hyperparameters of the one or more models, other characteristics of the models, or some combination thereof. In one or more embodiments, the model data store 290 is a component of the database 115.

### Multimodal Prediction of Peripheral Arterial Disease Risk

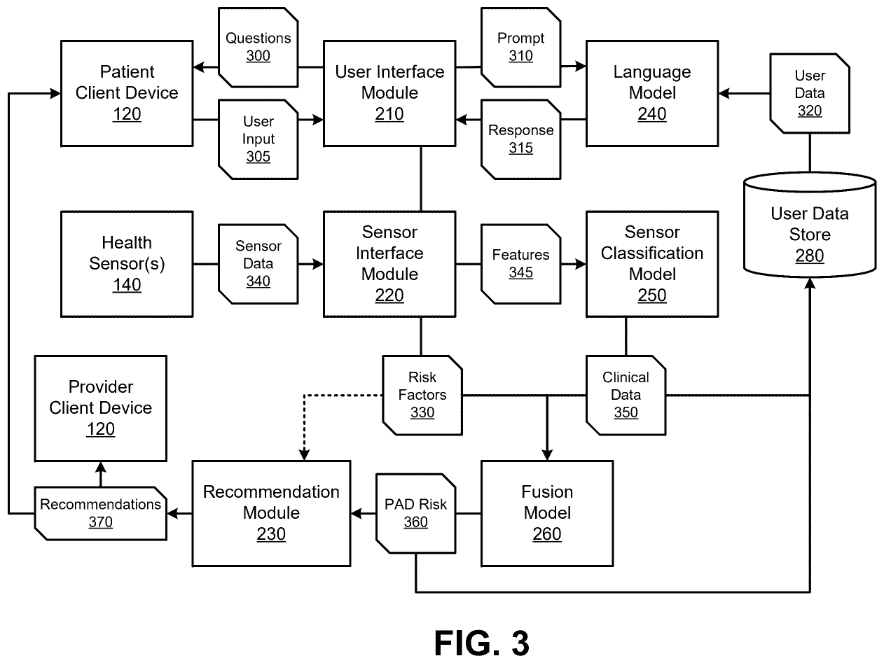

FIG. 3 is an illustrative flowchart of the multimodal prediction of PAD risk by the analytics system, according to one or more embodiments. The illustrative flowchart describes the interplay between components of the analytics system 110 as described in FIG. 2 with components in the networked computing environment 100 in FIG. 1. Although the analytics system 110 with its various components is described as performing the multimodal PAD risk prediction and/or the generation of personalized recommendations based on the results, in other embodiments, any of the steps may be disparately distributed across different entities. For example, the functionality of certain components of the analytics system 110 may be distributed onto an application programming interface being run on one or more client devices. It may also be understood to one of ordinary skill that the steps need not be performed in a single session, and may rather be split over multiple sessions or instances.

The patient client device 120 answers a dynamic questionnaire by the analytics system 110. The patient client device 120 may log onto a session with the analytics system 110 to aid in predicting PAD risk and/or identifying recommended treatments. When the patient client device 120 logs on, there may be an option on the presented user interface to request an updated PAD risk prediction. Upon selection, the user interface module 210 may automatically trigger a dynamic questionnaire to obtain updated information on the patient's health status. In some embodiments, there may be another option in the user interface to engage in the dynamic questionnaire, which upon selection would trigger the dynamic questionnaire.

The user interface module 210 presents one or more questions 300 on the user interface on the patient client device 120 querying about the patient's health status. In response, the patient client device 120 provides user input 305, which may be responsive to the questions 300. The user interface module 210 creates a prompt 310 including the user input 305 with one or more instructions to analyze the user input 305 and/or to generate follow-on content. The user interface module 210 serves the prompt 310 to the language model 240 for execution. The language model 240 may be tuned to performing NLP tasks related to generating the dynamic questionnaire. The language model 240 may further refer to the user data 320 previously stored in the user data store 280 relating to the current patient. For example, the dynamic questions generated by the language model 240 may refer to the patient's past medical history. The response 315 of the language model 240 to the prompt 310 is provided back to the user interface module 210. The user interface module 210 parses the prompt 310 to identify follow-on questions 300 to transmit to the patient client device 120 for presentation on the user interface.

The user interface module 210 may include a number of objectives to achieve in the dynamic questionnaire. As the user interface module 210 parses the user input 305 and/or the response 315, the user interface module 210 may identify patient-specific risk factors 330. If all objectives are hit, then the user interface module 210 may end the dynamic questionnaire. If there are certain objectives that, after a certain number of repeated clarification questions, were missed, the user interface module 210 may identify those objectives as issues for a healthcare provider to follow up on. The remaining objectives may be transmitted to the provider client device 120, e.g., in a report with the result of any analyses performed by the analytics system 110.

The health sensors 140 sense and record sensor data 340 that is provided to the sensor interface module 220. As noted elsewhere, in some embodiments, the health sensors 140 may be directly in communication with the analytics system 110. In other embodiments, the health sensors 140 may be in direct communication with the patient client device 120 which transmits the sensor data 340 to the sensor interface module 220 of the analytics system 110. The sensor interface module 220 extracts features 345 from the sensor data 340 to input into the sensor classification model 250. In one or more embodiments, the sensor classification model 250 is patient-agnostic, i.e., configured in the same manner when inputting features for one patient versus another. The sensor classification model 250 outputs clinical data 350 based on the input features 345.

The fusion model 260 inputs the risk factors 330 and/or the clinical data 350 (e.g., which may include diagnostic imaging data such as computed tomography angiography, magnetic resonance angiography, ultrasound, etc.) to perform multimodal PAD risk prediction. In some embodiments, a patient opts to decline responding to the questionnaire. In such cases, the analytics system 110 may identify any other known risk-factors, e.g., from a previously conducted questionnaire by the user interface module 210 or by a healthcare provider. The fusion model 260 inputs the risk factors 330 and the clinical data 350 to output the PAD risk prediction 360. The PAD risk prediction 360 may indicate whether the patient is at risk of PAD or not and/or a category of the PAD risk. The risk factors 330, the clinical data 350, the PAD risk prediction 360, or some combination thereof may be stored in the user data store 280 as user data, e.g., for subsequent recall and use by the analytics system 110.

The recommendation module 230 inputs the PAD risk prediction 360 to generate one or more recommendations 370 to address any PAD risk. In some embodiments, the recommendation module 230 may further input the risk factors 330, clinical data 350, other user data 320, or some combination thereof for further tailoring the recommendations 370 to the particular patient. The recommendations 370 may include treatment recommendations (e.g., if the PAD risk prediction 360 indicates the patient at risk of PAD) or preventive recommendations (e.g., if the PAD risk prediction 360 does not indicate the patient presently being at risk of PAD). The recommendations 370 may also include follow-on screening steps, e.g., to be performed by a healthcare provider, by the patient, or by one or more medical devices in communication with the analytics system 110. In one or more embodiments, the recommendation module 230 may further leverage a language model to identify the recommendations 370 from a list of available recommendations. The recommendation module 230 may further tailor recommendations to the healthcare provider's preferences.

The recommendations 370 are provided to the patient client device 120 and/or the provider client device 130. For example, in one paradigm, the recommendations 370 include treatment recommendations that are directly presented to the patient client device 120. In another paradigm, the treatment recommendations are presented to a healthcare provider, e.g., via the provider client device 130, for approval prior to transmitting to the patient client device 120. In yet other paradigms, the recommendations 370 may include control signals for controlling operation of one or more of the health sensors 140 and/or any other medical device that may aid in sensing of biometric signals or treatment of PAD (e.g., an at-home point-of-care device controlling the dispensing of medication).

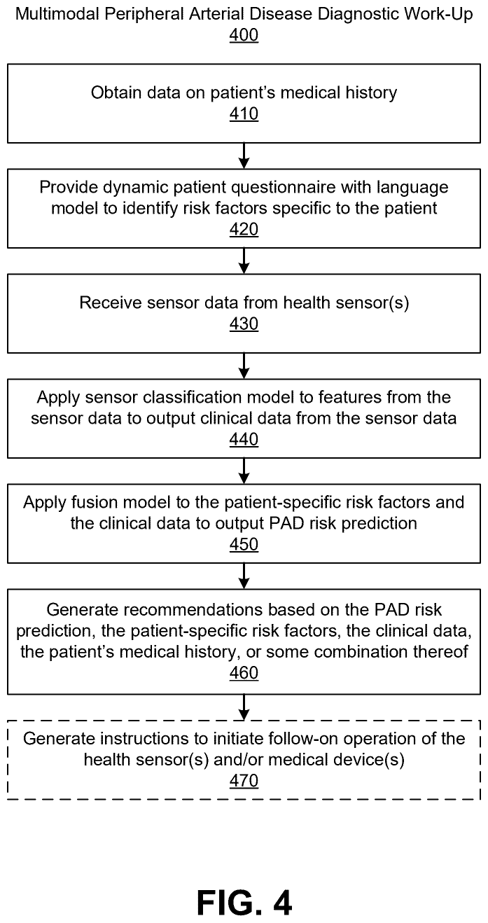

FIG. 4 is a method flowchart of the multimodal prediction of PAD risk and/or the generation of personalized recommendations by an analytics system, according to one or more embodiments. Although the analytics system (e.g., the analytics system 110 of FIG. 1 and/or FIG. 2) is described as performing the multimodal PAD risk prediction and/or the generation of personalized recommendations based on the results, in other embodiments, any of the steps may be performed by different entities. In other embodiments, the process may include additional, fewer, or different steps than those listed, e.g., including functionality described elsewhere throughout the disclosure. It may also be understood to one of ordinary skill that the steps need not be performed in a single session, and may rather be split over multiple sessions or instances.

In one or more embodiments, the analytics system obtains 410 data on a patient's medical history. The patient's medical history may be obtained from a third-party system, e.g., from a hospital computing server storing records on the patient, subject to the patient's authorized release of such data. In one or more example implementations, an application executed on the patient's client device recognizes the patient logging into the application. If the patient has not already done so, the application may invite the user to authorize the application to connect to an electronic health records system that stores the patient's health data, for retrieval of records for use in the PAD risk prediction. If the patient's health data is available in a connected database, the data may be read into the application.

The analytics system provides 420 a dynamic questionnaire with a language model to identify risk factors specific to the patient. The language model may be a machine-learning model trained to perform natural language processing (NLP) tasks, specifically relating to querying the patient to identify risk factors for PAD. In some embodiments, rule-based techniques may be used to convert patient data from a form available in the records system to a standard format. Alternatively, machine-learning techniques (such as application of large language models) may be used to interpret and parse the patient's healthcare data. In leveraging the language model, the analytics system may iteratively generate prompts for execution by the language model, outputting responses that may be parsed to provide the dynamic questionnaire to the patient. For example, the user interface may prompt the user to enter information such as a patient's complete history including present illness, past medical history, social history, medication, and symptoms include presence of lower extremity wounds or leg pain with exertion. Information may be entered into the application using text and/or voice entries that are converted to text. In instances of voice entries, the client device may include an acoustic sensor for recording audio signals pertaining to the patient's speech. The analytics system may leverage a voice-to-text recognition algorithm to convert the speech audio signal into speech text. The analytics system may parse inputs from the user, e.g., in response to the dynamic questionnaire, or responses from the language model in order to identify the patient-specific risk factors. In other embodiments, e.g., if the patient opts out of participating in the dynamic questionnaire, the analytics system may obtain patient-specific risk factors from prior session(s) or from an electronic health records system.

The analytics system receives 430 sensor data from one or more health sensors. The health sensors may be wearable devices for measuring biometric signals. The health sensors may be operated by the patient, or by a healthcare provider. The health sensors may measure signals including plethysmography data (e.g., via cuff devices coupled to different limbs of the patient's body), pulse rate, blood oxygen saturation, ultrasonic or other imaging data, blood volumetric flow, etc. In one or more embodiments, if diagnostic test results are not already available, the analytic system may initiate the sensing by the one or more health sensors. In some embodiments, the patient may utilize a pair of wearable devices that wirelessly couple to their client device for collecting sensor data for ABI evaluation. The ABI results may be automatically evaluated using rule-based on machine learning techniques. For example, an ABI ratio below 0.90 may indicate a high chance that the patient has hemodynamically significant arterial disease and may result in a recommendation for diagnostic arteriogram with surgical intervention if needed. Furthermore, a wearable device may collect data for an arterial ultrasound duplex. In one or more embodiments, the user interface presented on the patient's device by the analytics system may empower the patient to directly order the devices through the application and facilitate shipping to the user. As described above the analytics system may collect data from the wearable device and process the sensor data.

The analytics system applies 440 a sensor classification model to features of the sensor data to output clinical data from the sensor data. In one or more embodiments, the sensor classification model may leverage a function to regress the clinical data from the features. In some embodiments, the sensor classification model is a machine-learning model trained to output clinical data predictions based on the features. The clinical data may include metrics or characteristics on the heart or blood flow.

The analytic system applies 450 a fusion model to the patient-specific risk factors and/or the clinical data to output the PAD risk prediction for the patient. By leveraging information from the risk factors and the clinical data, the fusion model performs multimodal prediction of the PAD risk for the patient. The multimodal nature of the prediction expands the tailored prediction to the patient. For example, diagnosing purely based on clinical evidence screens against a distribution targeting an entire population of patients, such that the discrimination cutoffs would be generalized to the entire population. However, such generalization opens the door for missed diagnoses, if the patient doesn't quite align with the generalization. In some embodiments, the fusion model may be configured to input the patient-specific risk factors and/or the sensor data to output the PAD risk prediction. The PAD risk prediction may comprise a binary prediction (e.g., indicating whether the patient is at risk or not of PAD), a multiclass prediction (e.g., indicating whether the patient is at one of a plurality of risk categories for PAD), or a scalar value prediction (e.g., indicating a risk level for PAD).

The analytics system generates 460 recommendations based on the PAD risk prediction, the patient-specific risk factors, the clinical data, the patient's medical history, or some combination thereof. The analytics system may generate the recommendations leveraging a language model. The analytics system may generate treatment recommendations, follow-on investigative steps, instructions to control operation of health sensors and/or medical devices, or some combination thereof. In one example, the patient may be prescribed a specific medical therapy. Alternatively, the patient may be automatically referred for a lower extremity arteriogram procedure. The analytics system may generate a report indicating results of the analyses and/or the personalized recommendations. The report may be provided directly to the patient, and/or to a healthcare provider.

In some embodiments, the analytics system may generate 470 instructions to initiate follow-on operation of the one or more health sensors and/or one or more medical devices. With the health sensors, the instructions may cause the health sensors to initiate automated sensing to capture additional sensor data. With the medical devices, the instructions may cause the medical device to initiate automated therapies, e.g., dosing medication, scheduling a procedure, or performing/scheduling an autonomous or semi-autonomic robotic procedure.

In one or more embodiments, the analytics system may generate instructions for performing a surgical procedure. In such embodiments, the analytics system may generate an arterial map indicating precise visualization of the patient's arterial system. The analytics system may annotate the arterial map, e.g., leveraging vision-based models, to identify relevant features in the arterial map, including pathological regions such as stenoses, occlusions, or aneurysms, enabling highly targeted treatment planning and execution. The analytics system may further leverage the language model, referencing a template of steps for the surgical procedure, to generate the instructions. The instructions may instruct, e.g., how to obtain arterial access, how to manipulate guidewires, catheters, or any other medical device to arrive at a treatment site, how to perform the treatment at the treatment site with the medical device(s), etc. The arterial map and/or the instructions can be utilized either by a human surgeon or an autonomous robotic surgical system designed to perform endovascular procedures. In embodiments with a robotic surgical system aiding in the treatment procedure, the robotic surgical system may act in accordance with the instructions. Instructions may be produced in a natural language or be compiled to a domain specific language or machine binary for a targeted robotics system. For example, the robotic surgical system may obtain arterial access and independently manipulate guidewires and catheters within the arterial network to deliver treatments such as angioplasty, stenting, or atherectomy. The system may leverage real-time imaging, computational modeling, and advanced machine learning algorithms to ensure accurate navigation and precise deployment of therapeutic devices. Boundary conditions and sensor signal ranges and tolerances can be defined to allow the robotic system to execute movement with feedback from sensors during surgery. By automating aspects of vascular intervention, the system enhances procedural precision, reduces operator variability, and improves overall patient outcomes while maintaining safety and efficiency.

### Example Generation of Treatment Recommendations

FIG. 5 is an example treatment recommendation workflow, according to one or more embodiments. Here, a patient has a lifestyle limiting claudication 510 (leg pain with exertion, no wounds or pain at rest) being evaluated initially with ABI<0.90, the treatment plan may initially involve risk factor modification 520. If there is clinical evidence of infrainguinal disease 530, a trial of exercise and medical therapy 540 may be recommended. If the response is inadequate 540, additional tests may be performed with possible percutaneous therapy. Otherwise, if an adequate response 570 is observed, the patient may be recommended to follow up every six months. In an embodiment, the patient may reenter inputs into the user interface module 210 automatically (e.g., every six months). If symptoms remain unimproved or worsen and/or if the patient is still experiencing lifestyle limiting claudication, the patient's risk score may be automatically converted to 1, and the recommendation module 230 may operate according to the updated risk score to provide additional recommended treatments as described above.

### Additional Considerations

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a tangible non-transitory computer readable storage medium or any type of media suitable for storing electronic instructions and coupled to a computer system bus. Furthermore, any computing systems referred to in the specification may include a single processor or may be architectures employing multiple processor designs for increased computing capability.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

What is claimed is:

1. A method for predicting peripheral arterial disease (PAD) risk, the method comprising:
obtaining data on medical history of a user previously provided by the user;
providing, to a computing device associated with the user, a dynamic questionnaire comprising one or more questions generated by a language model based on input by the user;
receiving, from the computing device, input by the user in response to the dynamic questionnaire;
identifying one or more user-specific risk factors for PAD by parsing the input by the user;
receiving, from one or more health sensors, sensor data recorded by the one or more health sensors, wherein the sensor data includes biometric signals of the user measured by the health sensors;
applying a sensor classification model to the sensor data to output clinical data associated with a health state of the user;
applying a multimodal PAD risk prediction model to the identified user-specific risk factors and the clinical data to output a PAD risk prediction indicating whether the user is at risk for PAD;
generating one or more control signals to control operation of a health sensor or a medical device based on the PAD risk prediction and the user-specific risk factors; and
transmitting, to the health sensor or the medical device, the one or more control signals to cause operation of the health sensor for additional sensing or to cause operation of the medical device to provide therapy.

2. The method of claim 1, wherein providing the dynamic questionnaire comprises:
generating a prompt including one or more objectives indicating target information to be queried by the dynamic questionnaire and instructions to generate one or more questions targeting the objectives;
causing execution of the prompt by the language model to generate a response; and
identifying one or more questions to present in the dynamic questionnaire based on the response by the language model.

3. The method of claim 2, wherein generating the prompt comprises:
generating the prompt to further include the input by the user and instructions to generate one or more questions to identify additional details relating to the input by the user.

4. The method of claim 2, wherein generating the prompt comprises:
generating the prompt to further include the data on the medical history of the user and instructions to generate one or more questions to identify additional details relating to the medical history of the user.

5. The method of claim 1, wherein receiving the input by the user comprises:
receiving, from the client device, a speech signal captured by an acoustic sensor of the client device representing speech by the user; and
applying a voice-to-text recognition algorithm to convert the speech signal into speech text.

6. The method of claim 1, wherein identifying the one or more user-specific risk factors for PAD by parsing the input by the user comprises:
generating a prompt including the input by the user and instructions to identify any risk factors from a plurality of risk factors associated with PAD;
causing execution of the prompt by the language model to generate a response; and
identifying the one or more user-specific risk factors for PAD based on the response by the language model.

7. The method of claim 1,
wherein receiving the sensor data comprises receiving two sets of blood pressure data from two pressure cuff devices coupled to limbs of the patient, with one pressure cuff device coupled to an arm of the patient and another pressure cuff device coupled to an ankle of the patient; and
wherein applying the sensor classification model to the sensor data to output clinical data associated with the health state of the user comprises applying the sensor classification model to the two sets of blood pressure data to output an ankle-brachial index (ABI) value.

8. The method of claim 1, wherein receiving the sensor data comprises at least one of:
receiving plethysmography data;
receiving oximetry data;
receiving ultrasound duplex data;
receiving ultrasound Doppler data;
receiving computer tomography angiography data;
receiving magnetic resonance angiography data;
receiving fluoroscopy imaging data; and
receiving vascular imaging data.

9. The method of claim 1, wherein the sensor classification model is a machine-learning model trained by:
obtaining historical sensor data measured by health sensors in clinical environments and clinical data annotations from the sensor data;
generating training data with features derived from the historical sensor data and ground truth labels derived from the clinical data annotations; and
training the sensor classification model in a supervised manner with the training data.

10. The method of claim 1, wherein the multimodal PAD risk prediction model is a machine-learning model trained by:
obtaining historical PAD diagnoses by healthcare providers, clinical data from each PAD diagnosis, and any risk-factors identified in each PAD diagnosis;
generating training data with features derived from the clinical data and any identified risk-factors and ground truth labels derived from the PAD diagnoses; and
training the multimodal PAD risk prediction model in a supervised manner with the training data.

11. The method of claim 1, wherein generating the one or more personalized recommendations for the user based on the PAD risk prediction and the user-specific risk factors comprises:
generating a prompt including the PAD risk prediction, the user-specific risk factors, and a list of possible recommendations and instructions to identify the one or more personalized recommendations from the list of possible recommendations based on the PAD risk prediction and the user-specific risk factors; and
causing execution of the prompt by the language model to generate a response; and
identifying the one or more personalized recommendations based on the response by the language model.

12. The method of claim 11, further comprising:
receiving feedback from the user accepting or rejecting one or more of the personalized recommendations;
generating training samples with the feedback to the one or more personalized recommendations; and
retraining the language model to bias towards any recommendations accepted by the user and to bias away from any recommendations rejected by the user.

13. The method of claim 1, further comprising:
generating one or more personalized recommendations for the user based on the PAD risk prediction and the user-specific risk factors;
transmitting, to the computing device associated with the user, the one or more personalized recommendations for treating or mitigating PAD risk.

14. A non-transitory computer-readable storage medium storing instructions that, when executed by a processor, cause the processor to perform operations comprising:
obtaining data on medical history of a user previously provided by the user;
providing, to a computing device associated with the user, a dynamic questionnaire comprising one or more questions generated by a language model based on input by the user;
receiving, from the computing device, input by the user in response to the dynamic questionnaire;
identifying one or more user-specific risk factors for PAD by parsing the input by the user;
receiving, from one or more health sensors, sensor data recorded by the one or more health sensors, wherein the sensor data includes biometric signals of the user measured by the health sensors;
applying a sensor classification model to the sensor data to output clinical data associated with a health state of the user;
applying a multimodal PAD risk prediction model to the identified user-specific risk factors and the clinical data to output a PAD risk prediction indicating whether the user is at risk for PAD;
generating one or more control signals to control operation of a health sensor or a medical device based on the PAD risk prediction and the user-specific risk factors; and
transmitting, to the health sensor or the medical device, the one or more control signals to cause operation of the health sensor for additional sensing or to cause operation of the medical device to provide therapy.

15. The non-transitory computer-readable storage medium of claim 14, wherein providing the dynamic questionnaire comprises:
generating a prompt including one or more objectives indicating target information to be queried by the dynamic questionnaire and instructions to generate one or more questions targeting the objectives;
causing execution of the prompt by the language model to generate a response; and
identifying one or more questions to present in the dynamic questionnaire based on the response by the language model.

16. The non-transitory computer-readable storage medium of claim 14, wherein identifying the one or more user-specific risk factors for PAD by parsing the input by the user comprises:
generating a prompt including the input by the user and instructions to identify any risk factors from a plurality of risk factors associated with PAD;
causing execution of the prompt by the language model to generate a response; and
identifying the one or more user-specific risk factors for PAD based on the response by the language model.

17. The non-transitory computer-readable storage medium of claim 14,
wherein receiving the sensor data comprises receiving two sets of blood pressure data from two pressure cuff devices coupled to limbs of the patient, with one pressure cuff device coupled to an arm of the patient and another pressure cuff device coupled to an ankle of the patient; and
wherein applying the sensor classification model to the sensor data to output clinical data associated with the health state of the user comprises applying the sensor classification model to the two sets of blood pressure data to output an ankle-brachial index (ABI) value.

18. The non-transitory computer-readable storage medium of claim 14, wherein the sensor classification model is a machine-learning model trained by:
obtaining historical sensor data measured by health sensors in clinical environments and clinical data annotations from the sensor data;
generating training data with features derived from the historical sensor data and ground truth labels derived from the clinical data annotations; and
training the sensor classification model in a supervised manner with the training data.

19. The non-transitory computer-readable storage medium of claim 14, wherein the multimodal PAD risk prediction model is a machine-learning model trained by:
obtaining historical PAD diagnoses by healthcare providers, clinical data from each PAD diagnosis, and any risk-factors identified in each PAD diagnosis;
generating training data with features derived from the clinical data and any identified risk-factors and ground truth labels derived from the PAD diagnoses; and
training the multimodal PAD risk prediction model in a supervised manner with the training data.

20. A system comprising:
a processor; and
a non-transitory computer-readable storage medium storing instructions that, when executed by the processor, cause the processor to perform operations comprising:
obtaining data on medical history of a user previously provided by the user;
providing, to a computing device associated with the user, a dynamic questionnaire comprising one or more questions generated by a language model based on input by the user;
receiving, from the computing device, input by the user in response to the dynamic questionnaire;
identifying one or more user-specific risk factors for PAD by parsing the input by the user;
receiving, from one or more health sensors, sensor data recorded by the one or more health sensors, wherein the sensor data includes biometric signals of the user measured by the health sensors;
applying a sensor classification model to the sensor data to output clinical data associated with a health state of the user;
applying a multimodal PAD risk prediction model to the identified user-specific risk factors and the clinical data to output a PAD risk prediction indicating whether the user is at risk for PAD;
generating one or more control signals to control operation of a health sensor or a medical device based on the PAD risk prediction and the user-specific risk factors; and
transmitting, to the health sensor or the medical device, the one or more control signals to cause operation of the health sensor for additional sensing or to cause operation of the medical device to provide therapy.
